(19) United States
(12) Patent Application Publication
(10) Pub. No.: US 2023/0416844 A1
(43) Pub. Date: Dec. 28, 2023

(54) Methods To Analyze Methylomes In Tumor And Plasma Cell-Free DNA

(71) Applicant: The Trustees of Columbia University in the City of New York, New York, NY (US)

(72) Inventors: Regina Santella, Teaneck, NJ (US); Hui Zhou, New York, NY (US); Hui Chen Wu, New York, NY (US); Zhiguo Zhang, New York, NY (US)

(21) Appl. No.: 18/459,733

(22) Filed: Sep. 1, 2023

Related U.S. Application Data
(63) Continuation of application No. PCT/US2022/070998, filed on Mar. 7, 2022.
(60) Provisional application No. 63/157,479, filed on Mar. 5, 2021.

(51) Int. Cl. C12Q 1/6886; C12Q 1/686; G16H 50/20

(52) U.S. Cl. CPC C12Q 1/6886; C12Q 1/686; G16H 50/20; C12Q 2600/154; G01N 2800/7028

(57) ABSTRACT

Disclosed herein are methods for detecting methylation in cell-free polynucleotides and methods for detecting the presence of cancer in a subject.

### CROSS REFERENCE TO RELATED APPLICATIONS

This application is a continuation of International Application No. PCT/US2022/070998, which was filed on Mar. 7, 2022, which claims priority to U.S. Provisional Application No. 63/157,479, which was filed on Mar. 5, 2021, the contents of each of which are hereby incorporated by reference in its entirety.

### TECHNICAL FIELD

Disclosed herein are methods for detecting methylation in cell-free polynucleotides and methods for detecting the presence of cancer in a subject.

### BACKGROUND

Non-invasive methods of cancer detection are important for effective cancer treatment and auspicious patient outcomes. Different cancers and cell types have unique DNA methylation patterns. DNA methylation analysis is a strategy for cancer detection and characterization.

### SUMMARY

Disclosed herein are methods for detecting methylation in cell-free polynucleotides comprising: a) obtaining cell-free polynucleotides from a bodily sample from a subject, wherein the cell-free polynucleotides comprise single stranded DNA (ssDNA); b) ligating an ssDNA adaptor to the 3′ end of the ssDNA with an ssDNA ligase; c) converting the ssDNA from step c into double stranded DNA (dsDNA) with a DNA polymerase; d) ligating a dsDNA adaptor to the dsDNA from step c; e) denaturing the dsDNA from step d to obtain ssDNA; f) immunoprecipitating the ssDNA from step e using one or more antibodies against 5 mC and/or 5 hmC; g) amplifying the immunoprecipitated DNA; and h) sequencing the amplified DNA to detect the presence of and/or 5 hmC, thereby detecting methylation in the cell-free polynucleotides.

Also disclosed herein are methods for detecting the presence of cancer in a subject comprising: a) obtaining cell-free polynucleotides from a bodily sample from the subject and from a control subject known to be cancer-free; b) ligating an ssDNA adaptor to the 3′ end of the ssDNA with an ssDNA ligase; c) converting the ssDNA from step c into double stranded DNA (dsDNA) with a DNA polymerase; d) ligating a dsDNA adaptor to the dsDNA from step c; e) denaturing the dsDNA from step d to obtain ssDNA; f) immunoprecipitating the ssDNA from step e using one or more antibodies against 5 mC and/or 5 hmC; g) amplifying the immunoprecipitated DNA; h) sequencing the amplified DNA to detect the presence of 5 mC and/or 5 hmC, thereby detecting methylation in the cell-free polynucleotides of the subject and the cell-free polynucleotides of the control subject; and i) detecting cancer in the subject by comparing the presence of 5 mC and/or in the cell-free polynucleotides of the subject with that of the cell-free polynucleotides of the subject and control subject.

### BRIEF DESCRIPTION OF THE DRAWINGS

The file of this patent or application contains at least one drawing executed in color. Copies of this patent or patent application publication with color drawing(s) will be provided by the Office upon request and payment of the necessary fee.

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1A, FIG. 1B, FIG. 1C, and FIG. 1D illustrate an embodiment of the disclosed MeDIP-seq methods for detection of 5 mC in cfDNA. FIG. 1A shows an outline of an exemplary MeDIP-seq procedure. FIG. 1B shows a snapshot of MeDIP-seq of cfDNA in two samples (plasma DNA 002 and plasma DNA 005). FIG. 1C illustrates the number of unique mapped reads for each input and MeDIP-seq samples. FIG. 1D shows the MeDIP-seq read density surrounding transcription starting site (TSS) and transcription terminate sites (TTS) at three group of genes with indicated numbers of CpG dinucleotides.

FIG. 2A, FIG. 2B, FIG. 2C, and FIG. 2D illustrate an embodiment of the disclosed methods to detect DNA methylation in tumor DNA. FIG. 2A shows an outline of an exemplary procedure for detecting DNA methylation in tumor DNA. FIG. 2B illustrates a snapshot of MeDIP-seq in two DNA samples, 7511 (tumor) and 7512 (tumor adjacent region). FIG. 2C illustrates that unique mapped reads are high for our MeDIP-seq. FIG. 2D shows the MeDIP-seq read density surrounding transcription starting site (TSS) and transcription terminate sites (TTS) at three group of genes with indicated numbers of CpG dinucleotides.

FIG. 3 illustrates the impact of different amounts of input DNA on the quality of MeDIP-seq results. Three different amounts of input DNA (1.5 ng, 4.5 ng and ng) were used for MeDIPseq with mapped reads and unique mapped reads (filter-reads) shown in the top panel. The bottom panel shows the correlation of MeDIP-seq results from three different amounts of input DNA (left) with the input amounts in ng ssDNA and ill plasma samples shown at the right.

FIG. 4 illustrates the impact of different amounts of input DNA on the quality of MeDIP-seq results. Three different amounts of input DNA (9 ng, 18 ng and 70 ng) were used for MeDIP-seq with mapped reads and unique mapped reads (filter-reads) shown in the top panel. The bottom panel shows the correlation of MeDIP-seq results from three different amounts of input DNA (left) with the input amount in ng ssDNA and ill of plasma samples shown at the right.

FIG. 5A, FIG. 5B, and FIG. 5C illustrate the identification of cancer types based on Deep Neural Network (DNN). DNA methylation of cfDNA was analyzed from 78 samples consisting of three groups: subjects without cancer, patients with brain tumors, and patients with liver cancer. 46 samples were selected in the training set to build Deep Neural Network models and these models were used to predict the rest of the 32 samples (test cohort). The training cohort was used for the machine learning to build the models for DMR in cfDNA from each group of the samples, and the validation cohort was not used in the training and was used only for the evaluation of prediction. FIG. 5A depicts a schematic of the experimental design. The training cohort included 3 groups of cfDNA samples: subjects without cancer (“normal people”) (10), patients with brain tumors (20) and with liver cancer (16). FIG. 5B shows a heatmap of the top 600 DMRs from each group. The values in the heatmap are normalized against RPKM value. FIG. 5C illustrates the evaluation of the prediction of 32 samples in the validation cohort (13 brain tumor, 14 liver tumor, 5 normal) using an ROC curve. Area under each ROC curve is: brain=0.99, liver=0.96, normal=1, with the best probability cutoff for each group classification shown: normal=0.42, liver=0.35, brain=0.62.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of”.

The term “subject” as used herein is intended to mean any animal, in particular, mammals. Thus, the methods are applicable to human and nonhuman animals, although preferably used with mice and humans, and most preferably with humans. “Subject” and “patient” are used interchangeably herein.

Diagnosis of primary and reoccurring tumors often relies on invasive surgery. Non-invasive, early methods of detecting changes in DNA epigenetic marks from cell free DNA have been reported as an innovative tumor-specific diagnostic. However, during sample library preparation for sequencing, this method is hindered by the low abundance of cancer cell DNA that is necessary for accurately interrogating the methylome.

Provided herein are simplified, high-sensitivity methods of preparing and analyzing DNA methylome and hydroxylmethylome using plasma cell free DNA. The disclosed methods can couple single strand DNA ligation and antibody-specific DNA immunoprecipitation steps for amplifying library DNA for next-generation sequencing. Compared to existing methods, the disclosed methods can be more sensitive for the detection of methylome and hydroxylmethylome of plasma cell free DNA. Utilizing the disclosed methods advantageously allows non-invasive and early detection of cancer from cell free DNA, including difficult to detect central nervous system tumors.

Non-invasive screening methods are needed for the identification and diagnosis of cancer, including tumors of the central nervous system. Current non-invasive methods of analyzing cell free DNA epigenetic profiles lack the sensitivity to accurately detect cancer. Provided herein are sensitive methods to enhance and analyze cell free DNA for this purpose. The disclosed methods are able to detect specific epigenetic profiles by next-generation sequencing. As such, the disclosed methods have the potential to improve cancer diagnostics and treatment outcomes.

Early, non-invasive methods of detecting cancer hold great potential for improving precision oncology. DNA methylation patterns of cell free DNA can be used for early tumor detection. DNA methylation patterns are unique to cell type and tumor of origin. However, low abundance of circulating cell free DNA makes detection difficult. Methylome signatures in cell free DNA can be used for the detection of a variety of tumor types, including central nervous system tumors.

Disclosed herein are methods for detecting methylation in cell-free polynucleotides comprising:
a. obtaining cell-free polynucleotides from a bodily sample from a subject, wherein the cell-free polynucleotides comprise single stranded DNA (ssDNA);
b. ligating an ssDNA adaptor to the 3′ end of the ssDNA with an ssDNA ligase;
c. converting the ssDNA from step c into double stranded DNA (dsDNA) with a DNA polymerase;
d. ligating a dsDNA adaptor to the dsDNA from step c;
e. denaturing the dsDNA from step d to obtain ssDNA;
f immunoprecipitating the ssDNA from step e using one or more antibodies against 5 mC and/or 5 hmC;
g. amplifying the immunoprecipitated DNA; and
h. sequencing the amplified DNA to detect the presence of 5 mC and/or 5 hmC, thereby detecting methylation in the cell-free polynucleotides.

In some embodiments, the cell-free polynucleotides further comprise dsDNA. The method can further comprise a step of denaturing the dsDNA in the cell-free polynucleotides to obtain ssDNA, after step a and before step b.

Suitable bodily samples include blood, plasma, serum, urine, saliva, mucosal excretions, sputum, stool and tears. In some embodiments, the bodily sample is a plasma sample.

In some embodiments, the subject is a mammal.

The immunoprecipitating can be performed with one or more antibodies against 5 mC and 5 hmC. In some embodiments, the immunoprecipitating is performed with one or more antibodies against 5 mC. In some embodiments, the immunoprecipitating is performed with one or more antibodies against 5 hmC.

The amplifying can be performed by PCR.

Disclosed herein are methods for producing a methylated DNA immunoprecipitation sequencing (MeDIP-seq) library from plasma cell-free DNA (cfDNA), the method comprising: a) denaturing the plasma cfDNA to obtain single strand DNA (ssDNA); b) ligating a ssDNA adaptor to the 3′ end of the ssDNA; c) converting the ssDNA from step b into double stranded DNA (dsDNA); d) ligating a dsDNA adaptor to the dsDNA from step c; e) immunoprecipitating DNA using antibodies against 5 mC and/or 5 hmC; and f) amplifying the immunoprecipitated DNA.

The disclosed methods can be used to determine the DNA methylome and hydroxylmethylome using plasma cell free DNA from a sample of interest. In some embodiments, the sample is obtained from a subject's blood or plasma. In some embodiments the subject has cancer. Cancers capable of detection include, but are not limited to, brain tumors or liver cancer.

A range of blood or plasma volumes can be obtained for analysis. Suitable plasma volumes include, for example, 0.030 ml to 0.5 ml. In some embodiments, the plasma cell-free DNA is extracted from the sample obtained from the subject's blood or plasma. Extraction can be performed by any method known in the art. In some embodiments, the methods are performed using 1.5 ng to 70 ng of cfDNA.

Methods of performing the various reaction steps can be performed by any protocol known in the art. In some embodiments, ligation reactions are performed using DNA ligase enzymes. For example, the ssDNA adapter can be ligated to the 3′ end of the ssDNA with a ssDNA ligase. In some embodiments, the ssDNA is converted into dsDNA with a DNA polymerase.

Immunoprecipitation with antibodies against 5 mC and/or 5 hmC can be performed by any suitable protocol known in the art. In some embodiments, the methods comprise immunoprecipitating DNA using antibodies against 5 mC. In some embodiments, the methods comprise immunoprecipitating DNA using antibodies against 5 hmC. In some embodiments, dsDNA is immunoprecipitated.

The immunoprecipitated DNA can be amplified by any protocol known in the art. In some embodiments, the DNA is amplified by polymerase chain reaction (PCR).

In some embodiments, the methods of producing an MeDIP-seq library from plasma cell-free DNA (cfDNA) comprise:
a) obtaining a sample of blood from a subject;
b) extracting plasma cfDNA from the sample;
c) denaturing the plasma cfDNA to obtain single strand DNA (ssDNA);
d) ligating an ssDNA adaptor to the 3′ end of the ssDNA with an ssDNA ligase;
e) converting the ssDNA from step d into double stranded DNA (dsDNA) with a DNA polymerase;
f) ligating a dsDNA adaptor to the dsDNA from step e;
g) immunoprecipitating DNA using antibodies against 5 mC and/or 5 hmC; and
h) amplifying the immunoprecipitated DNA by polymerase chain reaction (PCR) to prepare the library.

Also provided are methods for detecting the presence of cancer in a subject comprising:
a. obtaining cell-free polynucleotides from a bodily sample from the subject and from a control subject known to be cancer-free;
b. ligating an ssDNA adaptor to the 3′ end of the ssDNA with an ssDNA ligase;
c. converting the ssDNA from step c into double stranded DNA (dsDNA) with a DNA polymerase;
d. ligating a dsDNA adaptor to the dsDNA from step c;
e. denaturing the dsDNA from step d to obtain ssDNA;
f immunoprecipitating the ssDNA from step e using one or more antibodies against 5 mC and/or 5 hmC;
g. amplifying the immunoprecipitated DNA;
h. sequencing the amplified DNA to detect the presence of 5 mC and/or 5 hmC, thereby detecting methylation in the cell-free polynucleotides of the subject and the cell-free polynucleotides of the control subject; and
i. detecting cancer in the subject by comparing the presence of 5 mC and/or in the cell-free polynucleotides of the subject with that of the cell-free polynucleotides of the subject and control subject.

In some embodiments, the cell-free polynucleotides further comprise dsDNA. The methods can further comprise a step of denaturing the dsDNA in the cell-free polynucleotides to obtain ssDNA, after step a and before step b.

Suitable bodily samples include blood, plasma, serum, urine, saliva, mucosal excretions, sputum, stool and tears. In some embodiments, the bodily sample is a plasma sample.

In some embodiments, the subject is a mammal.

The methods can detect central nervous system (CNS) cancer/tumor, colon cancer, liver cancer and pancreatic cancer. In some embodiments, the methods can detect a CNS cancer/tumor. In some embodiments, the CNS cancer/tumor is glioblastoma (GBM).

Detecting cancer can be achieved by employing Deep Neural Network (DNN).

In some embodiments, the detecting cancer comprises identifying a cancer type.

The immunoprecipitating can be performed with one or more antibodies against 5 mC and 5 hmC. In some embodiments, the immunoprecipitating is performed with one or more antibodies against 5 mC. In some embodiments, the immunoprecipitating is performed with one or more antibodies against 5 hmC.

The amplifying can be performed by PCR.

Also provided herein are methods for detecting a presence of cancer in a subject, the method comprising: detecting 5 mC or 5 hmC in a cfDNA sample; and diagnosing cancer in the subject by comparing the 5 mC or 5 hmC in the sample with the presence or absence of the 5 mC or 5 hmC in a sample known to be cancer-free.

DNA nucleotides can be modified with different functional groups. 5-methylcytosine (5 mC) is a methylated form of the DNA base cytosine. 5-hydroxymethylcytosine (5 hmC) is a hydroxymethylated form of the DNA base cytosine. Enzymes responsible for polymerization and transcription of nucleic acids can produce signatures when replicating or transcribing methylated DNA bases such as 5 mC or 5 hmC. These signatures, such as a 5 mC or a 5 hmC, can be detected via DNA sequencing to identify methylation sites.

The disclosed methods can be used to detect cancer in a subject using a sample of interest. In some embodiments, the sample is obtained from a subject's blood or plasma. In some embodiments the subject has cancer. Cancers capable of detection include, but are not limited to, brain tumors, liver cancer, tumors from central nerve system (CNS). In some embodiments, the cancer is a primary or recurrent glioblastoma (GBM) tumors.

A range of blood or plasma volumes can be obtained for analysis. Suitable plasma volumes include, for example, 0.030 ml to 0.5 ml. In some embodiments, the plasma cell-free DNA is extracted from the sample obtained from the subject's blood or plasma. Extraction can be performed by any method known in the art. In some embodiments, the methods can be performed on 1.5 ng to 70 ng of cfDNA.

In some embodiments, the methods of detecting the presence of cancer in a subject comprise:

obtaining a sample of plasma cell-free DNA (cfDNA) from a subject;

detecting the presence of 5 mC or 5 hmC in the cfDNA in the sample; and

diagnosing cancer in the subject by comparing the presence of 5 mC or 5 hmC when compared to the presence of 5 mC or 5 hmC in a sample known to be cancer-free.

Disclosed here are methods for detecting a presence of cancer in a subject, the methods comprising: detecting 5 mC or 5 hmC in a cfDNA sample, wherein the presence of the 5 mC or 5 hmC in the sample is indicative of the presence of cancer.

The 5 mC or 5 hmC can be detected by immunoprecipitation with antibodies against 5 mC or 5 hmC. In some embodiments, the methods comprise detecting in the cfDNA by immunoprecipitation with antibodies against 5 mC. In some embodiments, the methods comprise detecting 5 hmC in the cfDNA by immunoprecipitation with antibodies against 5 hmC.

The disclosed methods have the following advantages:
Provide high-sensitivity methods for analyzing DNA methylome and hydroxylmethylome using plasma cell free DNA.
Enhance detection of cell free DNA methylosome and hydroxylmethylome by antibody-specific immunoprecipitation
Processing of DNA methylation profiles in low amount cell free DNA.
Utilize single strand DNA ligase for sensitive detection of cell free DNA.
Simplify library preparation for next-generation sequencing.
The methods can be used for non-invasive early detection of cancer, such as various tumors, particularly glioblastoma (GBM), colon, and pancreatic cancers.

The methods herein can be used for analysis of DNA methylome and hydroxylmethylome using plasma cell free DNA. Among other benefits, these methods can be useful for non-invasive early detection of cancer. The methods herein have been developed through unique ways; libraries herein have been prepared for detection of DNA methylosome and hydroxylmethylome by next-generation sequencing.

Applications of the methods include: analysis of methylome using plasma cell free DNA, non-invasive cancer diagnoses, early detection of cancer, monitoring recurrent GBM and other cancers, monitoring tissue specific epigenetic profiles, and studying the epigenetic mechanisms underlying various cancers.

### EXAMPLES

The following examples are provided to further describe some of the embodiments disclosed herein. The examples are intended to illustrate, not to limit, the disclosed embodiments.

### Introduction

An early diagnosis of tumors is critical for improving treatment outcomes. Therefore, there has been extensive interest in the analysis of 5 mC and 5 hmC in plasma cell free DNA (cfDNA) for early tumor detection. Compared to genetic mutations, detection of 5 mC and 5 hmC offers several advantages. First, early studies indicate that epigenetic changes such as DNA methylation may proceed genetic mutations during cell transformation. Moreover, 5 mC and 5 hmC patterns are unique in each cell type. Therefore, 5 mC and 5 hmC patterns, if detected, are likely associated with cell origins of tumors. Second, because of low abundance of cfDNA released from cancer cells, it is very challenging to detect a limited amount of tumor specific mutations in cfDNA, which are largely from normal cells. Because 5 mC and 5 hmC are prevalent on the human genome and these epigenetic marks are dramatically altered in tumor cells, it may be easier to detect tumor-specific changes of 5 mC and 5 hmC in cfDNA. Indeed, several recent studies have reported that 5 mC and 5 hmC cfDNA signatures can be used for the detection of a variety of tumors. Even more surprising is a report this year that DNA methylomes in cfDNA can be used for the detection of CNS tumors. Because of the blood-brain barrier, tumor DNA released into blood may be even lower compared to other tumor types. This study could, for some cases, bypass the need of invasive surgery for the diagnosis of tumors from central nerve system (CNS) and therefore dramatically reduces risk and neurological morbidity of CNS tumor patients.

With the support from the Herbert Irving Comprehensive Cancer Center (HICCC), sensitive methods to analyze DNA methylomes in cfDNA and tumor DNA have been developed. These methods can also be used to analyze 5 hmC genome-wide. 5 mC and will be analyzed in cfDNA from patients of primary and recurrent GBM tumors to determine if 5 mC and/or 5 hmC unique signatures can be identified and used for the detection of CNS primary tumors as well as recurrent GBM tumors. These results can lead to early detection of CNS tumors and recurrent GBM tumor non-invasively, which should help to improve the treatment outcomes of this deadly disease. The proposed studies will help detection and diagnosis of GBM tumors non-invasively. Importantly, the study will help detect GBM recurrence using plasma cell free DNA, which is challenging to detect by imaging because of complications from the treatment induced lesions. Finally, the proposed studies in GBM tumors can be easily translated into early detection of other tumor types such as colon and pancreatic cancers in collaboration with other laboratories at HICCC.

### Example 1—Developing a Sensitive Method for Detecting 5 mC in cfDNA Genome Wide

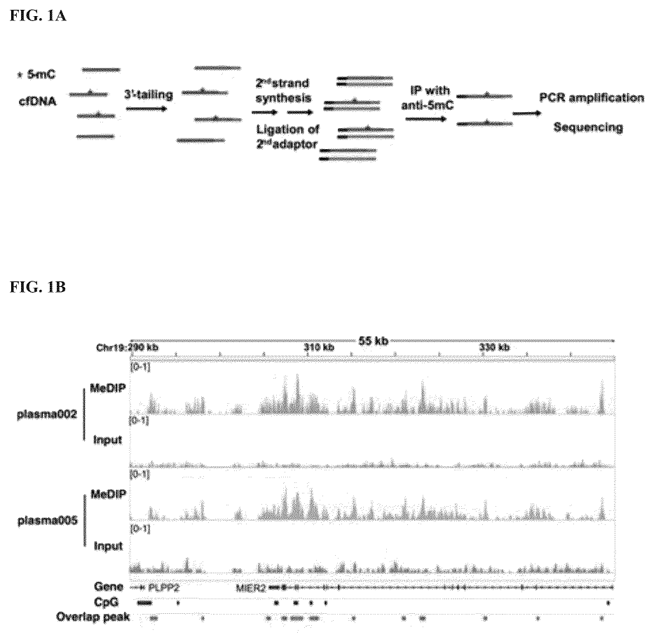

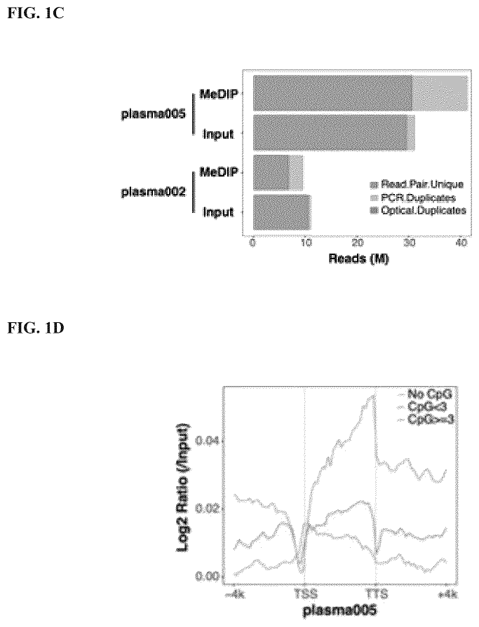

Plasma cell free DNA (cfDNA) is a mixture of DNA released from various tissues. The majority of cfDNA is about 100 nt single stranded DNA (ssDNA). Moreover, bisulfite sequencing, the traditional method for DNA methylation detection, is not reliable for low levels of cfDNA. Therefore, methylated DNA immunoprecipitation coupled with next-generation of sequencing (MeDIP-seq) was developed to analyze DNA methylation in cfDNA. We took advantage of our extensive experience preparing ssDNA libraries for next-generation sequencing and optimized MeDIP-seq procedures for detecting DNA methylation in low amount cfDNA (FIG. 1A). Briefly, to analyze DNA methylation in cfDNA, an adaptor was first ligated to the 3′ end of ssDNA using ssDNA ligase followed by converting ssDNA into dsDNA by a DNA polymerase. After the ligation of the second adaptor, a small fraction of DNA (10%) was saved as input, and the majority of DNA was denatured into ssDNA and subjected to immunoprecipitation using antibodies against 5 mC. The immunoprecipitated DNA as well as the input DNA were then amplified by PCR for library preparation and subsequent deep sequencing (FIG. 1A). Using this method, MeDIP-seq libraries were produced from cfDNA isolated from 2.5 ml human blood (FIG. 1B, FIG. 1C, and FIG. 1D). The library quality was excellent as the percentage of mapped reads was over 60% (FIG. 1C). Moreover, DNA methylation peaks were readily detected when comparing with the input samples (FIG. 1B). Finally, MeDIP-seq read density was depleted at TSS regions of genes with over 3 CpG dinucleotides, but was enriched at gene bodies and TTS of these genes compared to genes with less than 3 CpG dinucleotides. This distribution is consistent with the distribution of DNA methylation in cells, validating specificity of the MeDIP-seq for detection of 5 mC in cfDNA.

The same procedure can be followed to analyze 5 hmC by replacing antibodies against 5 mC with 5 hmC. Moreover, because of the utilization of ssDNA ligase for library preparation, which has been widely used to analyze ancient DNA, the current procedure is more sensitive for the detection of 5 mC and 5 hmC of cfDNA than any published methods.

### Example 2—Developing a Sensitive Method for Detecting 5 mC of Tumor DNA Genome Wide

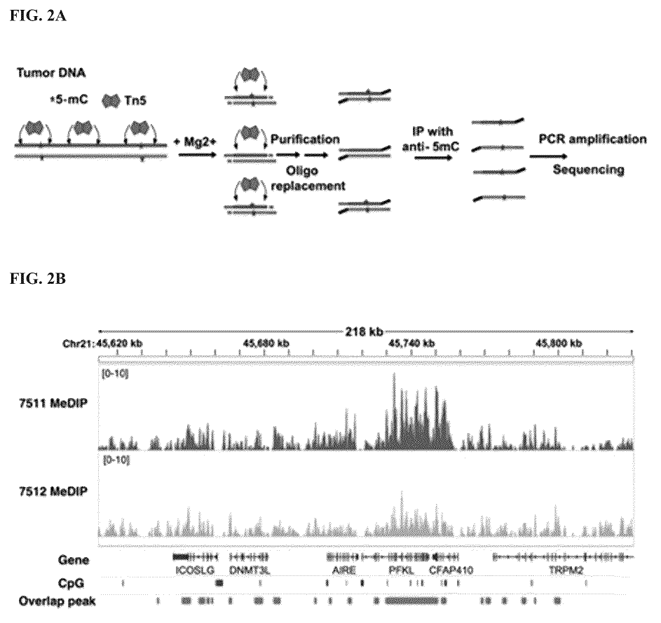

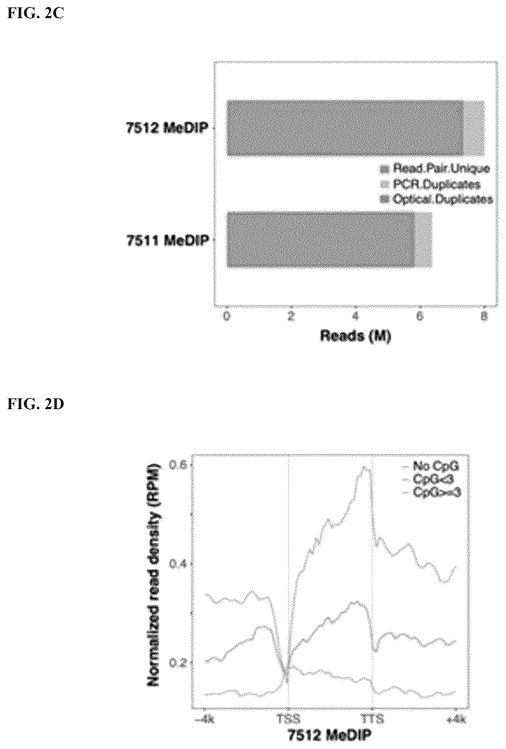

The majority of DNA isolated from tumor cells are large fragments of double stranded DNA (dsDNA). Therefore, to analyze DNA methylation on dsDNA from tumor samples, transposase Tn5 was utilized to fragment and tag dsDNA first (FIG. 2A). Briefly, 10 ng of dsDNA isolated from tumor samples was subjected to tagmentation by the Tn5 transposase. This enzyme can insert an adaptor into dsDNA in a sequence independent manner. As Tn5 transposase only covalently ligates one strand of the adaptor to target DNA, a different adaptor was ligated at the 3′ end through the oligo-replacement step. In this way, the DNA methylation pattern can be analyzed in a strand-specific manner. Following tagmentation, dsDNA was denatured, and methylated DNA was immunoprecipitated using antibodies against 5 mC. The enriched methylated ssDNA was amplified by PCR for library preparation and subsequent sequencing. Using this method, the DNA methylation of two samples, DNA isolated from a tumor (7511) and from its adjacent region (7512), was analyzed. As shown in FIG. 2B, MeDIP-seq peaks were readily identified. The unique mapped sequence reads, which measures data quality, was high (FIG. 2C). Finally, the distribution of MeDIP-seq density at TSS and TTS at genes with different CpG dinucleotide was consistent with published results on the distribution of DNA methylation at these genic regions, indicating that the disclosed MeDIP-seq was able to detect DNA methylation with high specificity.

### Example 3— Analysis of Liver and Brain Tumors

Plasma cell free DNA (cfDNA) is a mixture of DNA released from various tissues. The majority of cfDNA is about 100 nucleotides of single stranded DNA (ssDNA). Bisulfite sequencing, the traditional method for DNA methylation detection, is not reliable for low levels of cfDNA. Therefore, methods of performing methylated DNA immunoprecipitation coupled with next generation sequencing (MeDIP-seq) were generated to analyze DNA methylation in cfDNA. ssDNA libraries were prepared for next-generation sequencing and MeDIP-seq procedures were optimized for detecting DNA methylation in low amounts cfDNA.

Using this optimized method, reliable MeDIP-seq results were generated from cfDNA isolated from 30 μl to 0.5 ml plasma samples. The impact of different amounts of input cfDNA on the quality of MeDIP-seq results were analyzed in two samples. In one sample from a subject without cancer (referred to as “normal person” herein), MeDIP-seq data was obtained from as low as 1.5 ng of plasma cfDNA, which is equivalent to 45 μl of the plasma sample (FIG. 1). The MeDIP-seq datasets from three different amounts of samples showed good correlations, with higher quantities of DNA resulting in higher quality MeDIP-seq data (FIG. 1).. Significantly higher amounts of cfDNA was isolated from a brain tumor patient (FIG. 2). MeDIP-seq results from three different amounts of cfDNA from this sample all generated high quality data with high correlations (FIG. 2). In this sample, because of the presence of high amount of cfDNA in plasma, 30 μl plasma was enough to generate high quality MeDIP-seq results. Together, these results indicate that the amount of cfDNA isolated from different people varies. However, this analysis also indicates that reliable MeDIPseq results can be obtained from as low as 30 μl plasma, while varying among different samples.

This method was applied and DNA methylation of cfDNA (isolated from ml of plasma) was analyzed from 78 samples (including 33 patients with brain tumors, patients with liver cancer, and 15 from normal people). MeDIP-seq datasets of 10 samples from normal (normal), 20 from brain tumor (brain), and 15 from liver cancer (liver) were chosen as the training cohort to identify differentiated methylated regions (DMR) in each group through machine learning. To reduce the influences from the diversity of individual samples, 80% of the training cohort was randomly sampled 10 times in a balanced way for each group (normal, brain and liver). In each (from 1 to 10) subset cohort, each group's specific DMRs in a one-versus-other way were identified and the top 100 hyper- and 100 hypo-DMRs for each group were selected.

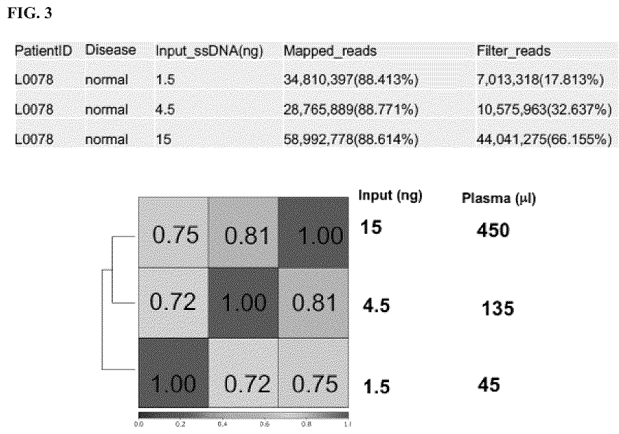

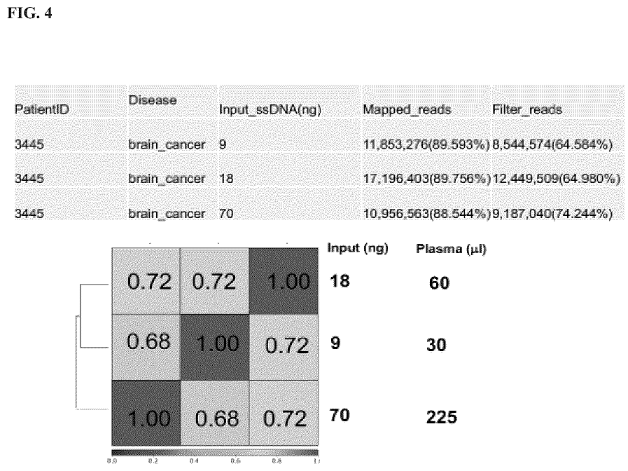

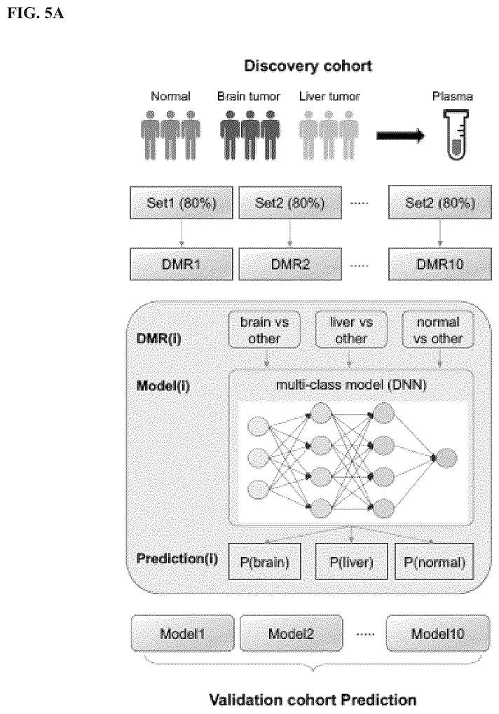

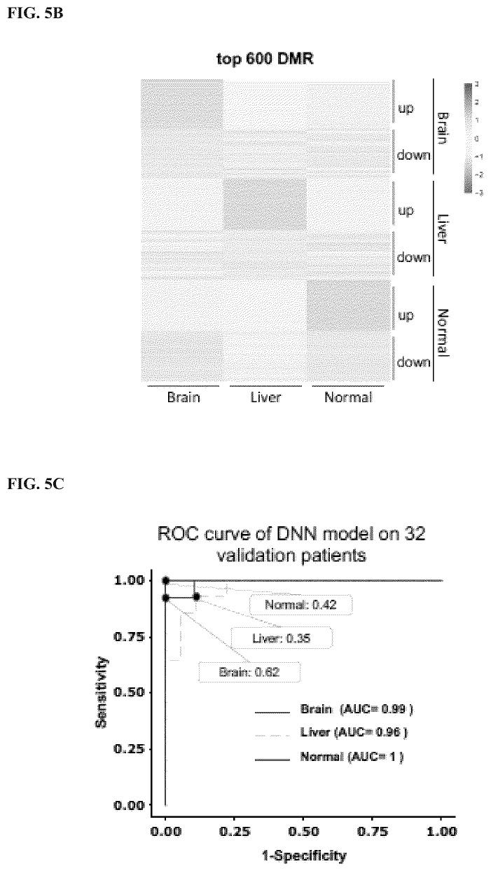

In total, 600 DMRs were selected for the machine learning model training. Then the 5 mC RPKM on 600 DMRs were used as input (FIG. 3B) and a Deep Neural Network (DNN) model of 3 layers (64, 32, 3 nodes on each layer) was trained from the subset training cohort to build a 3-classification model (FIG. 3A, FIG. 3B). In total, subset machine learning models were built based on the training cohort (FIG. 3A, FIG. 3B). Each model was then used to predict the rest of the samples that were not used in the machine learning (total 32 samples with 13 samples from brain tumor, 14 samples from liver tumor, 5 from normal people). 10 different prediction probabilities were generated for each sample, with the average from the 10 subset models shown in FIG. 3C. As shown in FIG. 3C, the rest of the samples were identified successfully. Together, these studies indicate that liver and brain tumors can be identified by analyzing DNA methylation of plasma DNA from these patients. Because of the blood-brain barrier, it is well accepted that much less DNA from brain tumors will be released into the blood than other tumor types. Therefore, the current method of analyzing DNA methylation of plasma cfDNA will also be able to identify other tumor types.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### EMBODIMENTS

The following list of embodiments is intended to complement, rather than displace or supersede, the previous descriptions.

Embodiment 1. A method for detecting methylation in cell-free polynucleotides comprising:
a. obtaining cell-free polynucleotides from a bodily sample from a subject, wherein the cell-free polynucleotides comprise single stranded DNA (ssDNA);
b. ligating an ssDNA adaptor to the 3′ end of the ssDNA with an ssDNA ligase;
c. converting the ssDNA from step c into double stranded DNA (dsDNA) with a DNA polymerase;
d. ligating a dsDNA adaptor to the dsDNA from step c;
e. denaturing the dsDNA from step d to obtain ssDNA;
f immunoprecipitating the ssDNA from step e using one or more antibodies against 5 mC and/or 5 hmC;
g. amplifying the immunoprecipitated DNA; and
h. sequencing the amplified DNA to detect the presence of 5 mC and/or 5 hmC, thereby detecting methylation in the cell-free polynucleotides.

Embodiment 2. The method of embodiment 1, wherein the cell-free polynucleotides further comprise dsDNA, and wherein the method further comprises a step of denaturing the dsDNA in the cell-free polynucleotides to obtain ssDNA, after step a and before step b.

Embodiment 3. The method of embodiment 1 or 2, wherein the bodily sample is selected from the group consisting of blood, plasma, serum, urine, saliva, mucosal excretions, sputum, stool and tears.

Embodiment 4. The method of embodiment 3, wherein the bodily sample is a plasma sample.

Embodiment 5. The method of any one of the previous embodiments, wherein the subject is a mammal.

Embodiment 6. The method of any one of the previous embodiments, wherein the immunoprecipitating is performed with one or more antibodies against 5 mC.

Embodiment 7. The method of any one of the previous embodiments, wherein the amplifying is performed by PCR.

Embodiment 8. A method for detecting the presence of cancer in a subject comprising:
a. obtaining cell-free polynucleotides from a bodily sample from the subject and from a control subject known to be cancer-free;
b. ligating an ssDNA adaptor to the 3′ end of the ssDNA with an ssDNA ligase;
c. converting the ssDNA from step c into double stranded DNA (dsDNA) with a DNA polymerase;
d. ligating a dsDNA adaptor to the dsDNA from step c;
e. denaturing the dsDNA from step d to obtain ssDNA;
f immunoprecipitating the ssDNA from step e using one or more antibodies against 5 mC and/or 5 hmC;
g. amplifying the immunoprecipitated DNA;
h. sequencing the amplified DNA to detect the presence of 5 mC and/or 5 hmC, thereby detecting methylation in the cell-free polynucleotides of the subject and the cell-free polynucleotides of the control subject; and
i. detecting cancer in the subject by comparing the presence of 5 mC and/or in the cell-free polynucleotides of the subject with that of the cell-free polynucleotides of the subject and control subject.

Embodiment 9. The method of embodiment 8, wherein the cell-free polynucleotides further comprise dsDNA, and wherein the method further comprises a step of denaturing the dsDNA in the cell-free polynucleotides to obtain ssDNA, after step a and before step b.

Embodiment 10. The method of embodiment 8 or 9, wherein the bodily sample is selected from the group consisting of blood, plasma, serum, urine, saliva, mucosal excretions, sputum, stool and tears.

Embodiment 11. The method of embodiment 10, wherein the bodily sample is a plasma sample.

Embodiment 12. The method of any one of embodiments 8-11, wherein the subject is a mammal.

Embodiment 13. The method of any one of embodiments 8-12, wherein the cancer is selected from the group consisting of central nervous system (CNS) cancer/tumor, colon cancer, liver cancer and pancreatic cancer.

Embodiment 14. The method of embodiment 13, wherein the cancer is a CNS cancer/tumor.

Embodiment 15. The method of embodiment 14, wherein the CNS cancer/tumor is glioblastoma (GBM).

Embodiment 16. The method of any one of embodiments 8-15, wherein detecting cancer is achieved by employing Deep Neural Network (DNN).

Embodiment 17. The method of any one of embodiments 8-16, wherein detecting cancer comprises identifying a cancer type.

Embodiment 18. The method of any one of embodiments 8-17, wherein the immunoprecipitating is performed with one or more antibodies against 5 mC.

Embodiment 19. The method of any one of embodiments 8-18, wherein the amplifying is performed by PCR.

What is claimed:

1. A method for detecting methylation in cell-free polynucleotides, the method comprising:
a. obtaining cell-free polynucleotides from a bodily sample from a subject, wherein the cell-free polynucleotides comprise single stranded DNA (ssDNA);
b. ligating an ssDNA adaptor to the 3′ end of the ssDNA with an ssDNA ligase;
c. converting the ssDNA from step c into double stranded DNA (dsDNA) with a DNA polymerase;
d. ligating a dsDNA adaptor to the dsDNA from step c;
e. denaturing the dsDNA from step d to obtain ssDNA;
f immunoprecipitating the ssDNA from step e using one or more antibodies against 5 mC and/or 5 hmC;
g. amplifying the immunoprecipitated DNA; and
h. sequencing the amplified DNA to detect the presence of 5 mC and/or 5 hmC, thereby detecting methylation in the cell-free polynucleotides.

2. The method of claim 1, wherein the cell-free polynucleotides further comprise dsDNA, and wherein the method further comprises a step of denaturing the dsDNA in the cell-free polynucleotides to obtain ssDNA, after step a and before step b.

3. The method of claim 1, wherein the bodily sample is selected from blood, plasma, serum, urine, saliva, mucosal excretions, sputum, stool, and tears.

4. The method of claim 3, wherein the bodily sample is plasma.

5. The method of claim 1, wherein the subject is a mammal.

6. The method of claim 1, wherein the immunoprecipitating is performed with one or more antibodies against 5 mC.

7. The method of claim 1, wherein the amplifying is performed by PCR.

8. A method for detecting the presence of cancer in a subject, the method comprising:
a. obtaining cell-free polynucleotides from a bodily sample from the subject and from a control subject known to be cancer-free;
b. ligating an ssDNA adaptor to the 3′ end of the ssDNA with an ssDNA ligase;
c. converting the ssDNA from step c into double stranded DNA (dsDNA) with a DNA polymerase;
d. ligating a dsDNA adaptor to the dsDNA from step c;
e. denaturing the dsDNA from step d to obtain ssDNA;
f immunoprecipitating the ssDNA from step e using one or more antibodies against 5 mC and/or 5 hmC;
g. amplifying the immunoprecipitated DNA;
h. sequencing the amplified DNA to detect the presence of 5 mC and/or 5 hmC, thereby detecting methylation in the cell-free polynucleotides of the subject and the cell-free polynucleotides of the control subject; and
i. detecting cancer in the subject by comparing the presence of 5 mC and/or in the cell-free polynucleotides of the subject with that of the cell-free polynucleotides of the subject and control subject.

9. The method of claim 8, wherein the cell-free polynucleotides further comprise dsDNA, and wherein the method further comprises a step of denaturing the dsDNA in the cell-free polynucleotides to obtain ssDNA, after step a and before step b.

10. The method of claim 8, wherein the bodily sample is selected from blood, plasma, serum, urine, saliva, mucosal excretions, sputum, stool, and tears.

11. The method of claim 10, wherein the bodily sample is plasma.

12. The method of claim 8, wherein the subject is a mammal.

13. The method of claim 8, wherein the cancer is selected from a central nervous system (CNS) cancer/tumor, colon cancer, liver cancer, and pancreatic cancer.

14. The method of claim 13, wherein the cancer is a CNS cancer/tumor.

15. The method of claim 14, wherein the CNS cancer/tumor is glioblastoma (GBM).

16. The method of claim 8, wherein detecting cancer is achieved by employing Deep Neural Network (DNN).

17. The method of claim 8, wherein detecting cancer comprises identifying a cancer type.

18. The method of claim 8, wherein the immunoprecipitating is performed with one or more antibodies against 5 mC.

19. The method of claim 8, wherein the amplifying is performed by PCR.
